(19) United States
(12) Patent Application Publication
(10) Pub. No.: US 2020/0194125 A1
(43) Pub. Date: Jun. 18, 2020

(54) Method and System for Tracking, Storing, and Processing Data to Identify Risk Factors and Predict Health-Related Conditions

(71) Applicants: Andrew Adolphus, Brooklyn, NY (US); Jordan S. Rathus, Brooklyn, NY (US); Spencer A. Rathus, Surfside, FL (US); Lois Fichner-Rathus, Surfside, FL (US)

(72) Inventors: Andrew Adolphus, Brooklyn, NY (US); Jordan S. Rathus, Brooklyn, NY (US); Spencer A. Rathus, Surfside, FL (US); Lois Fichner-Rathus, Surfside, FL (US)

(21) Appl. No.: 16/687,433

(22) Filed: Nov. 18, 2019

Related U.S. Application Data
(63) Continuation-in-part of application No. 15/166,157, filed on May 26, 2016, now abandoned.
(60) Provisional application No. 62/167,252, filed on May 27, 2015.

(51) Int. Cl. G16H 50/30; G16H 50/70; G16H 80/00; G06F 9/54; H04W 4/029

(52) U.S. Cl. CPC G16H 50/30; G16H 50/70; H04W 4/029; G06F 9/542; G16H 80/00

(57) ABSTRACT

A system and method are disclosed comprising a mobile platform that tracks and correlates user-generated data and application-sourced data to ascertain environmental and personal risk factors or causes related to a user's physiological and mental health symptoms and conditions. The mobile platform alerts users when risk factors or causes are identified and predicts occurrences of the symptoms and conditions. Further, the platform enables users to observe selected symptoms and health conditions within a chosen venue or geographic range or location, anonymously identify other users within that venue or range, and communicate with other users within a venue, range, or location. The venue or range may also be given a score that indicates the presence or prevalence of various symptoms or health conditions.

### CROSS-REFERENCE TO RELATED APPLICATION

The present application claims priority to U.S. application Ser. No. 15/166,157, filed on May 26, 2016, which claims the benefit of U.S. Provisional Application Ser. No. 62/167,252, filed on May 27, 2015, both of which are hereby incorporated by reference herein in their entirety, including any figures, tables, equations, or drawings.

### FIELD

The systems, devices, and methods disclosed herein relate generally to processing and analyzing data. More particularly, the systems, devices, and methods relate to tracking and predicting medical risk factors.

### BACKGROUND

Society is increasingly becoming more health conscious. Advances in science have identified some relationships between environmental factors and individual health risks in an individual's overall immediate and future health. As a result, individuals seek individualized information on health risks factors.

Various mobile platforms attempt to provide users with information regarding allergies, migraines, and other health conditions. Some allergy related applications use the Global Positioning System (“GPS”) and public records of pollen counts to alert users of possible allergic risks. Further, various online and mobile platforms allow users to search for relevant causes and diagnoses related to specific symptoms. Yet still other mobile platforms alert users to allergic reactions associated with various venues, such as restaurants. A problem in many of these systems is that they rely on the user to input information regarding the user's symptoms prior to providing notices of relevant diagnoses or risk factors. For an individual, it can be time consuming to input their medical information. Further, the user may not be aware of pertinent information to include. Although known mobile platforms can include diagnoses, such diagnoses can be generic and not tailored to the unique environmental and health risks of the user.

Accordingly, a need exits in the art for a system and method that requires minimal initial input from a user to receive individualized health-related notifications. A system is needed that automatically integrates user-generated data, including current physiological data, with data from service providers to generate notices concerning the causes or risk factors related to health conditions and predict health risks. It would be further advantageous for users to be able to communicate with other users with similar conditions in a given venue or geographic range or location.

Yet another problem with the various mobile platforms that attempt to provide users with information regarding health conditions is that each mobile platform utilizes a different interface. As a result, users are required to learn various different interfaces which can be difficult.

Accordingly, a need exists in the art for a common interface for a user to receive individualized information on health risks factors from various mobile platforms regarding health conditions.

### SUMMARY

A system and method for identifying risk factors and predicting health related conditions is provided. Unlike known systems that track for a specific health purpose (i.e., migraine-tracking, allergy-tracking, and food diary applications), the system and methods disclosed can track various types of symptoms, including but not limited to, migraines, allergies, moods, psoriasis flare-ups, acne, stress, seasonal affective disorder, sexual dysfunctions, gastrointestinal problems, insomnia, and other sleep disorders, such as nightmares. Users can either select from a predetermined list of symptoms or health conditions and/or input their own.

### BRIEF DESCRIPTION OF THE DRAWINGS

The detailed description makes reference to the accompanying figures wherein:

FIG. 1 illustrates an exemplary network diagram;

FIG. 2 is a block diagram depicting a computer system architecture for implementing the mobile platform in accordance with various embodiments;

FIG. 3 is a flowchart depicting an exemplary process according to the preferred embodiment;

FIG. 4 is a flowchart depicting an exemplary process involving asthma symptoms;

FIG. 5 is a flowchart depicting an exemplary process executed by the mobile platform in accordance with the principles disclosed herein;

FIG. 6 is a flowchart depicting an exemplary process in which the mobile platform stores multiple datasets;

FIG. 7 illustrates an exemplary graph of the information stored in a user profile in accordance with the preferred embodiment;

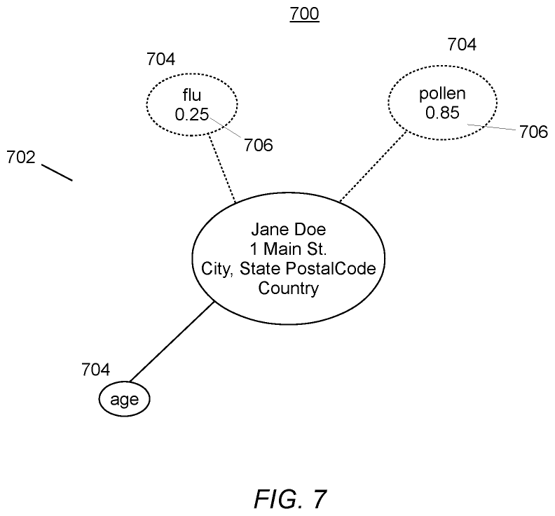

FIG. 8 illustrates an exemplary screen diagram showing a user interface; and

Other objects, features, and characteristics, as well as methods of operation and functions of the related elements of the structure and the combination of parts, will become more apparent upon consideration of the following detailed description with reference to the accompanying drawings.

### DETAILED DESCRIPTION

A detailed illustrative embodiment is disclosed herein. However, techniques, methods, processes, systems and operating structures in accordance with the disclosure may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure.

None of the terms used herein, including “terminal,” “one button,” “provider,” and “module” are meant to limit the application of the invention. The terms are used to illustrate the preferred embodiment and are not intended to limit the scope of the invention. Similarly, the use of these terms is not meant to limit the scope or application of the invention, as the invention is versatile and can be utilized in many applications, as will be apparent in light of the disclosure set forth herein. The following presents a detailed description of the preferred embodiment with reference to the figures.

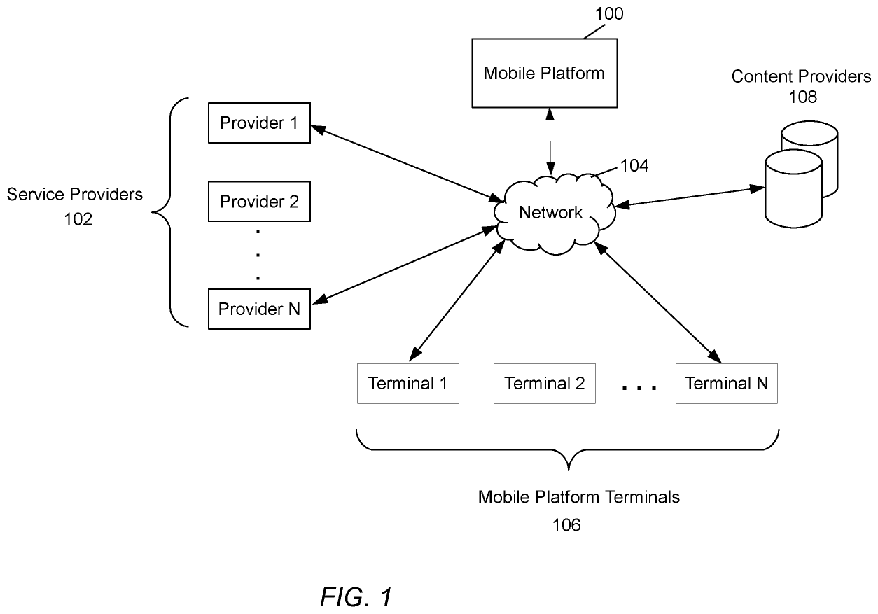

Referring initially to FIG. 1, shown is an exemplary network diagram of mobile platform 100. Mobile platform 100 can be implemented on hardware or a combination of hardware and software. In the preferred embodiment, the techniques disclosed herein are implemented in a software environment such as an operating system or in an application running on an operating system. This software can include, but is not limited to, resident software, firmware, etc., or is implemented on a cloud-based or visualized network system.

Mobile platform terminals 106 communicate over network 104 with mobile platform 100. Mobile platform terminals 106 are preferably configured to receive, process, store, and transmit information. The information can be visual, auditory, tactile (i.e., when a smartphone vibrates), or even olfactory, as when a device converts olfactory information into a digital format and transmits. Exemplary mobile platform terminals include, but are not limited to, a mobile telephone, cellular telephone, smart telephone, laptop computer, netbook, personal digital assistant (PDA), smart watches, optical head-mounted displays (OHMDs), smart clothing, smart refrigerators or any other computing device suitable for network communication.

Network 104 can be a local area network (LAN), a wide area network (WAN), the Internet, cellular networks, satellite networks or any other network that permits the transfer and/or reception of data to and/or from mobile platform 100. The data transmitted to or from mobile platform 100 through network 104 can be transmitted and/or received utilizing standard telecommunications protocol or standard networking protocol. In the preferred embodiment, the system utilizes Transmission Control Protocol/Internet Protocol (TCP/IP) and network 104 is the Internet. Other examples of protocols for transmitting and/or receiving data include but are not limited to Voice Over IP (VOIP) protocol, Short Message Service (SMS), and Global System for Mobile Communications (GSM). Network 104 is capable of utilizing one or more protocols of mobile platform 100. Furthermore, network 104 can translate to or from other protocols to one or more protocols of mobile platform terminals 106. Therefore, a user can seamlessly transition from one device to another and continue to track, store, and process data to identify risk factors and predict health-related conditions from mobile platform 100.

Service providers 102 communicate over network 104 with mobile platform 100. Health providers can use service providers 102 to communicate information on the services that they can provide to users on mobile platform terminals 106. Health providers comprise businesses and groups that could provide assistance to users after mobile platform 100 identifies a health risk or medical condition. Exemplary health providers include, but are not limited to, fitness clubs, emergency care providers, yoga studios, tea shops, stress relief programs, gluten free businesses, and medical specialists. In the present embodiment, a health provider creates a service profile on mobile platform 100. The health provider service profile can include a location, approved health insurance plans, and services provided by the health provider. Therefore, mobile platform 100 can recommend relevant health providers for risk factors and/or medical conditions identified for a user.

Mobile platform 100 can communicate over network 104 with content providers 108. Content providers 108 can include but are not limited to the U.S. Food and Drug Administration Recalls, Market Withdrawals, & Safety Alerts; Centers for Disease Control and Prevention; National Weather Service; National Centers for Environmental Information; the World Health Organization; and any other service that provides domestic and international health related news, alerts, and other information available through network 104. Further, mobile platform 100 can scrape the content of various websites, including but not limited to, a municipality website. Thereafter, the information (e.g., local health related notices) can be utilized in addition to information from other content providers 108 when determining the notifications to send to users.

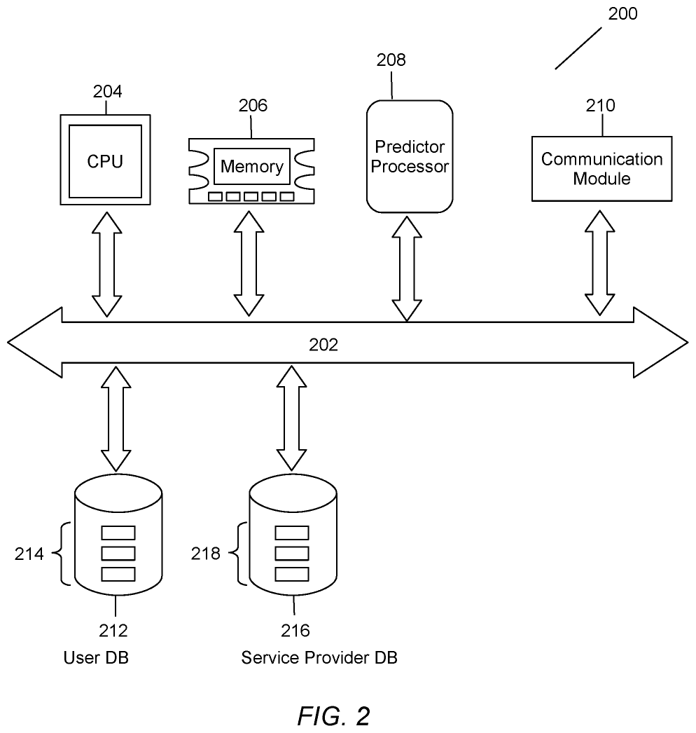

Referring now to FIG. 2, shown is an exemplary block diagram depicting a computer system architecture for implementing mobile platform 100. At least one computer processing unit (CPU) 204 is interconnected to bus 202. At least one memory 206 is interconnected to CPU 204 through bus 202. Communication Module 210 allows mobile platform 100 to communicate through a network with mobile platform terminals, service providers, and content providers.

The computer system architecture further includes at least one database. The databases described below can store data over one or multiple databases. In the preferred embodiment, user database 212 comprises at least one user profile 214. User profile 214 securely stores various information about a user, including but not limited to age, medical history, allergies, potential risk factors, and family medical history. The information stored in user profile 214 can be directly inputted by the user, or mobile platform 100 can predict the information using predictor processor 208. In the preferred embodiment, predictor processor 208 utilizes various weighted factors to determine the possible health risk factors.

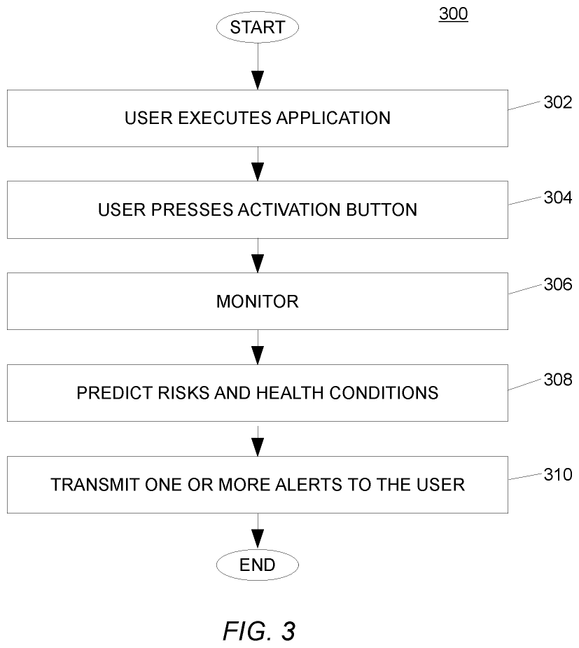

FIG. 3 depicts a flowchart 300 representing a user experience in accordance with the preferred embodiment. First, in step 302, the user downloads or executes an application on the user's mobile platform terminal. Unlike other similar applications, which require that the user input data prior to providing relevant information, the application in this embodiment has one single “button” that the user presses or touches upon the initial opening of the application. In step 304, the user presses the “button.”

Next in step 306, the mobile platform stores a timestamp in the user profile. Thereafter, the mobile platform monitors various sources of information, including but not limited to, user-selected (or later inputted) symptoms, environmental conditions, information from content providers, and application-sourced data. In step 308, the mobile platform predicts risk factors for a user profile based on the information monitored in step 306.

Thereafter, in step 310, the mobile platform notifies the user of the risk factors and health-related conditions identified in step 308. The data can be displayed in various ways, including pie charts, graphs, charts, maps, annotated maps, and maps displaying the locations of other users.

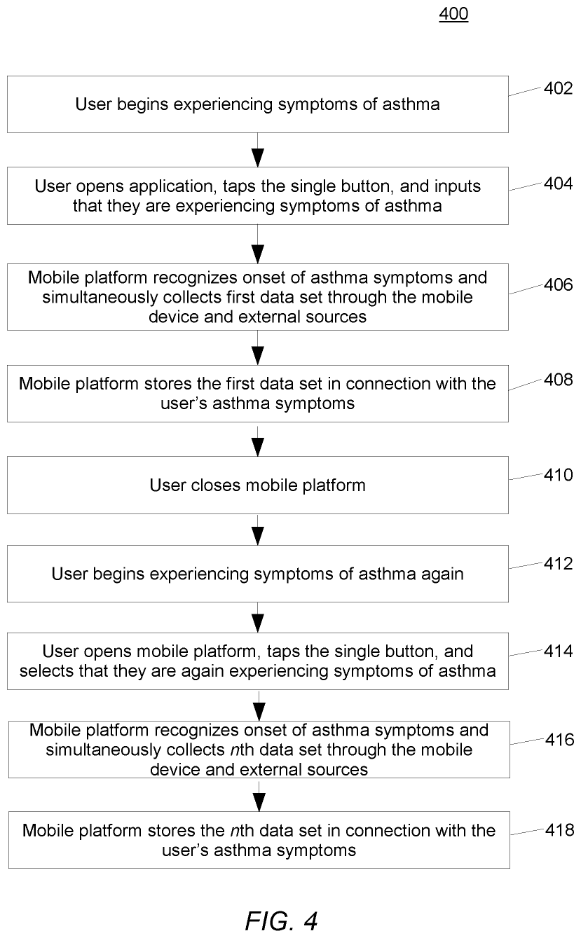

FIG. 4 displays a flow chart illustrating an embodiment for determining the risk factors based on the information monitored. In this example, the risk factors will be determined for a user experiencing symptoms of asthma. FIG. 4 shows how the asthma symptom data is collected over time. In step 402, the user begins experiencing symptoms of asthma, such as shortness of breath, coughing that will not stop, tightened neck and chest muscles, and severe wheezing.

Once a user begins experiencing these symptoms, the user proceeds to step 404. In step 404, a user accesses the mobile platform and taps an access button. Pressing this button prompts the user to input the asthmatic symptoms the user is experiencing, which is stored in the mobile platform for future selection.

In step 406, the mobile platform recognizes the onset of asthma symptoms in the user, and begins collecting a first set of data to associate with the asthma symptoms. The mobile application repeats data cycling from a number of sources automatically, but only elects to store specific data once the mobile platform recognizes the onset of a user's symptoms. In this example, this first set of data is collected from the mobile device and external sources. In particular, the mobile platform will collect time of day and GPS data from the mobile device, including the geographical location and elevation of the user at the time the user taps the access button. The mobile platform also collects data from external sources, such as the National Weather Service, local weather resources, local and national centers for disease control, local environmental sources, news sources (e.g., sources reporting local events impacting atmospheric conditions such as a forest fire within a defined proximity to the user that is emitting harmful gases, waterline contamination or high level of toxins in drinking water, etc.), or may even link to user-supplied sources such as an electronic thermometer or commonly known Internet-linked home climate-controlled systems that collect various local environmental conditions within or outside a user home. Specifically, for asthma, this additional data may include temperature, humidity, barometric pressure, pollen count, and the presence of smog or other atmospheric particulates in the area, etc. The mobile platform can collect data from various additional external sources depending on the desired elements measured. For example, specifically for an asthma analysis, a heart rate monitor or fitness band worn by the user and remotely connected to the mobile device may be used. The heart rate monitor or fitness band can be useful for analyzing a user's heart rate and physical activity level, and the obtained data can be collected, analyzed, recorded, etc. at defined intervals for the particular user to establish baseline parameters specific to the user. In step 408, the mobile platform records and analyzes the data it collected and stores it in connection with the asthmatic symptoms the user is experiencing. In this example, the mobile platform can display the first data set to the user such that the user is apprised of the certain elements that may impact the symptoms a user experiences.

Next, in step 410, the user closes the application. It is contemplated that the application can passively operate by recording predetermined elements even after the user closes the application.

As shown in step 412, the user begins experiencing symptoms of asthma again, prompting the user to access the mobile platform, in step 414, by pressing the access button and selecting the ailment the user is experiencing, in this particular example, symptoms associated with asthma. In the present embodiment, each time a user experiences certain ailments, the aforementioned recordings are maintained and the analysis is conducted so that new data points and experiences are maintained from the second time to the nth time, thereby improving the accuracy of the platform. Specifically, in step 416, the mobile platform again recognizes the onset of asthma symptoms and begins collecting the aforementioned data for the nth time. This nth data set is generally made of up of the same types of data as the first data set. However, the specific values of the data (i.e. the actual temperature, how much pollen is in the air, etc.) have the potential to vary while others may remain the same. In step 418, the mobile platform again stores the nth data set in connection with the user's asthma symptoms.

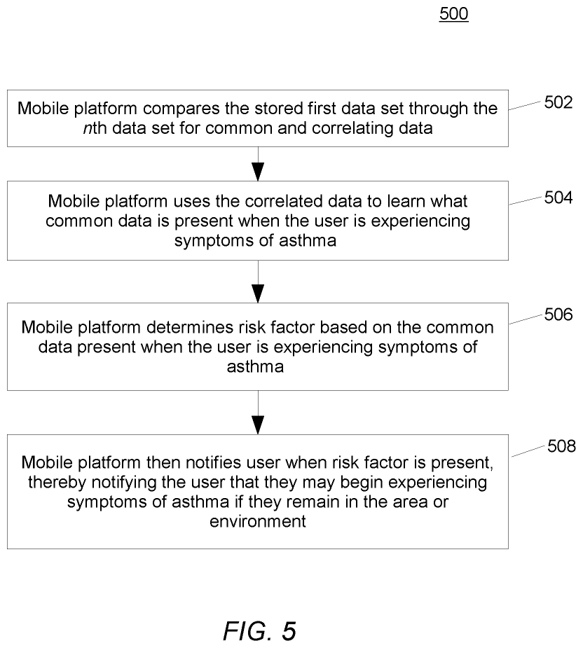

FIG. 5 represents the analysis of the collected data for determining the risk factor associated with the asthmatic symptoms. The learning and analysis of the information may be performed by various analytic algorithms known in the art; however, in the preferred embodiment, the mobile platform of the present invention aggregates and clusters the various datasets of the user with datasets of other users. The datasets are variables describing characteristics of clustered communities that are analyzed based on known parameters culminated from pertinent studies targeted for specific health symptoms, including but not limited to migraines, allergies, moods, psoriasis flare-ups, acne, stress, seasonal affective disorder, sexual dysfunctions, gastrointestinal problems, insomnia, and other sleep disorders, such as nightmares. The platform automatically integrates user-generated data, including current physiological data, with data from service providers to generate notices concerning the causes or risk factors related to health conditions and predict health risks. Each user is identified as a single input source defined to create a plurality of communities wherein each community comprises one or more crowd members who exhibit similar characteristics, such as common genetic traits. For example, a community may comprise users observed to have similar biases to certain environmental conditions based on user-supplied or mobile platform-supplied traits. The learning of the mobile platform comprises a probabilistic graphical model comprising a plurality of sub-models whereby a message passing schedule is facilitated for fast, efficient training with web-scale data. The system is further capable of interpreting noise or false reads controlled by the probabilistic settings. Probability distributions representing belief about the data set variables may be initialized to default values and updated during platform learning update processes as data sets are observed by the mobile platform.

The description sets forth the functions of examples below and the sequence of steps for constructing and operating the examples. However, the same or equivalent functions and sequences may be accomplished by different examples. In step 502, the mobile platform compares all of the stored data sets, established by the specific user, as each data sets relates to the asthma symptoms, from the first data set to the nth data set. Generally, the more data sets the mobile platform has collected, the more accurate the risk factor determination will be. While the data sets are supplied by users via accessing the mobile platform, the submitted data sets can be sent to the platform in various formats. For example, the platform may serve web pages publishing queries, and the users may have end user computing equipment enabling the users to submit answers to the published queries. While the use of web-based communications are the preferred method of obtaining data sets, the platform may publish the queries in other ways, for example, by sending email messages, and may receive answers in any suitable automated manner over a communications network.

At this stage, the mobile platform compares all of the stored data sets to establish commonalities and correlations between each of the data points. Based on these correlations, clusters of users can be formed. The virtual clusters of users are based on observed similar biases that the users generate in response to queries for data sets. However, depending on the ailment, the mobile platform is capable of analyzing a subset of data points, excluding certain data such as data points that fall well outside of a determined range by setting probability vectors, which are designed to reflect false data or another abnormality in the data obtained. Accuracy of the results may be assessed by comparison with ground truth data where that is available. For example, ground truth data may be established by a user device enables to transmit GPS data compared to user-inputted location data. Ground truth data may also be based on commonly accepted medical studies in the art. In step 504, the mobile platform utilizes the commonalities and correlations to establish common data established at the onset of the user experiencing symptoms of asthma. For exemplary purposes, if the data sets show a user beginning to experience symptoms of asthma when the user is physically located in a downtown area when the air temperature is recorded at 55 degrees Fahrenheit, with pollen count recorded at high levels based on data obtained from certain external sources, the mobile device, taking into account known studies related to asthma, learns that these factors have the potential to be the cause of the asthmatic symptoms in the user.

Next in step 506, the mobile platform utilizes these common data points to create a risk factor correlating to the common data points. Weighting of the common data points is based on the known risk factors specific to a particular concern. For example, with respect to asthma, certain identified particulates such as those associated with industrial pollution or particulates common to allergens are attributed a higher weigh than risk factors associated with sun exposure. When the common data points are present, the risk factor is triggered. However, when the common data points are not present, the risk factor will not be present.

In step 508, the mobile platform notifies the user when the risk factor is present. Since the mobile platform is constantly cycling through data from the mobile device and other external sources, the mobile platform can determine when the risk factor is present based on whether the common data points are present. Using the previous example, if the mobile platform determines that the user is located in a downtown area, the temperature is around 55 degrees Fahrenheit, and the pollen count is at high levels, the mobile platform knows the risk factors are present and notifies the user that the user may begin experiencing symptoms of asthma before the user actually experiences the symptoms. The system is also capable of analyzing data within a certain radius to alert the user to avoid a certain area so that the user can avoid experiencing the ailment. In this example, the user can decide whether to stay in the area or environment, or leave the area or environment to avoid the symptoms.

Area, temperature, and pollen count are three examples of what data points could point to a risk factor for asthma. However, the mobile platform is able to determine a number of potential causes of a number of different ailments. For example if a user is experiencing allergies, common allergy triggers that can be reported include airborne allergens, such as pollen, animal dander, dust mites and mold; food allergens, particularly peanuts, tree nuts, wheat, soy, fish, shellfish, eggs and milk; insect stings, such as from a bee or wasp; or medications, particularly penicillin or penicillin-based antibiotics. Certain risk factors may increase the impact of allergy including family history, asthma, or age. If a user is experiencing migraines, it is well known that while genetic attributes are a significant factor, lifestyle and environmental factors slightly outweigh genetic structure. In this example, such environmental factors are heavily weighted by the algorithm of the mobile platform. For example, the primary risk factors associated with migraines include sex, hormonal changes (birth control, menstrual cycle fluctuation), hormone replacement therapy, weight considerations, foods (e.g., aged cheeses, salty food, etc.), food additives (e.g., aspartame, MSG, etc.), alcohol consumption, caffeine consumption, use of various medications (e.g., oral contraceptives and vasodilators, impact of sensory stimuli (e.g., bright lights, loud sounds, and strong odors, all of which are primary aspects that can be shared via the mobile platform of the present invention), stress, physical exertion, environmental changes such as barometric pressure and humidity, sleep patterns, and family history.

The mobile platform and algorithm is particularly useful for constantly evolving viruses such as the influenza virus. New strains of influenza viruses appear regularly. If a user had influenza in the past, the user's body develops antibodies to fight that particular strain of the virus. Thus, if future influenza viruses are similar to those encountered by the user, those antibodies may prevent infection or lessen its severity. However antibodies against flu viruses encountered in the past do not protect the user from new influenza strains that can be very different immunologically from prior viruses. In this instance, in order to evaluate influenza concerns, the mobile platform considers risk factors including the user's age, living or working conditions (users in hospitals, nursing homes, or locations with unsanitary conditions are more likely to be exposed to the virus), weakened immune conditions (cancer treatments, anti-rejection drugs, long-term use of steroids, organ transplant, blood cancer or HIV/AIDS can weaken a user's immune system), chronic illness (lung diseases such as asthma, diabetes, heart disease, neurological or neurodevelopmental disease, an airway abnormality, and kidney, liver or blood disease, may increase risk of influenza complications), pregnancy, or weight concerns. The mobile platform is capable of analyzing additional known complications associated with the influenza virus such as pneumonia, bronchitis, asthma flare-ups, heart conditions, and ear infections.

In another example, the mobile platform interprets risk factors associated with cancer including a user's age, use of tobacco, sun exposure, radiation exposure, alcohol use, weight concerns, exposure to cancer-causing materials, and alcohol use. It is well known that cancer is primarily influenced by external factors, and a substantial majority of human cancers are the result of exposure to environmental agents and therefore preventable. Thus, the use of the mobile platform to evaluate and track the identified environmental risks is critical. Further, the evaluation and tracking of the interaction by the user with the mobile platform is likely to carve a decisive path to genetic analysis and research currently being conducted to clarify the known aspects of cancer and develop a cure thereto.

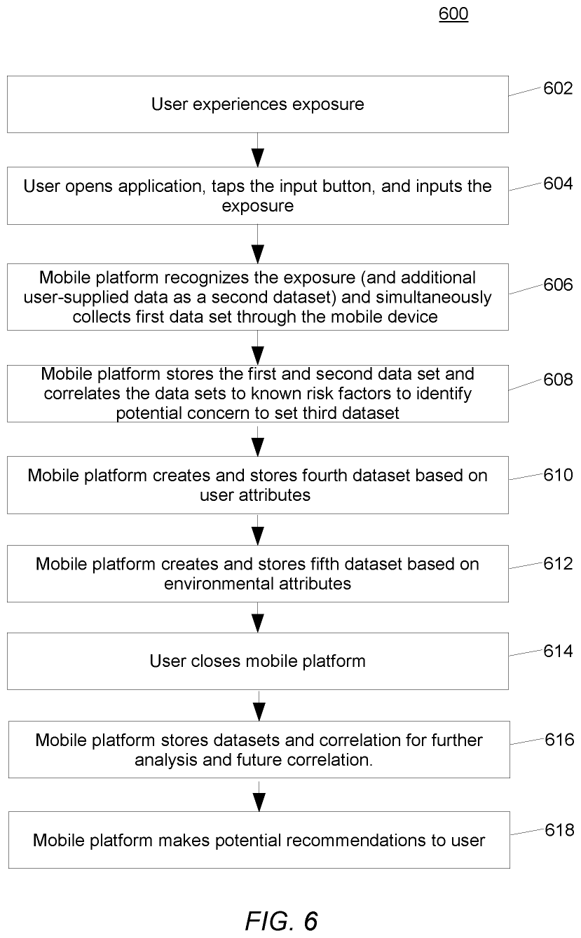

The mobile platform is further designed to consider and track user responses that may result in the reduction of cancer, including tracking of a user's diet. For example, in one embodiment of the present invention as depicted in FIG. 6, a user experiences an exposure in step 602, then accesses the mobile platform and taps an access button in step 604. Pressing this button prompts the user to input an exposure the user is experiencing, which is stored in the mobile platform for future selection. For example, a user may input weather exposure, including exposure to the sun on a particularly sunny day. Such information may be useful in tracking a user's exposure to cancer-causing agents including those common with skin cancer. This application may be particular useful in identifying a user's condition over an extended duration of exposure to a specific skin-causing cancer agent or a multitude of skin-causing cancer agents. The user may further input the user's location, such as close to the equator where the sun exposure is strong and long in duration. In addition, the user can input data confirming that the user has applied a specific SPF lotion on the particular area and applied the lotion within a time parameter. The user may also input information confirming the user was smoking a particular cigarette on the specified day. With reference to step 606, this particular user-inputted data set is stored in the mobile platform as second dataset for present and future analysis. With reference to step 608, to create a third subset of data, the mobile platform develops information matching the user entered data with known risk factors associated various ailments. In step 610, the mobile platform develops a fourth subset of data by analyzing information specific to the user such as genetic history, family history, dietary factors, etc. to establish and develop the user's genetic profile. In step 612, the mobile platform can develop a fifth subset of data by analyzing specific conditions developed by the mobile platform such as data obtained independent of user input. For example, while a user can provide the user's location, such as close to the equator where the sun exposure is strong and long in duration, the mobile platform can independently develop, explore, or confirm this information. Specifically, the mobile platform can access latitude and longitude coordinates and elevation specific to the mobile device thereby further confirming or narrowing the input supplied by the user. The mobile platform can also establish data points based on the duration the user remains at the identified location, thereby identifying the length and strength of the sun exposure for the specific input. Notably at this stage, the mobile platform may further expand the data point by accessing various Internet resources such as exploring the reported effectiveness, stated manufacturer effectiveness, or ingredients of the identified SPF lotion by interpreting the lotion's manufacturer webpage or other reported reviews on independent websites. The data point can further be developed based on the user's smoking specific to the type of cigarette smoked.

Based on these four subsets, the mobile platform can record this information for future use, analyze the information, and/or correlate the new datasets with prior data specific to the user, or as a fifth data subset, the mobile platform can assess other users' data to correlate same and develop any corresponding environmental agents that may impact the user or other users of the mobile platform. For example, the mobile platform can collect data from external sources, such as the National Weather Service, local weather resources, local and national centers for disease control, local environmental sources, news sources (e.g., sources reporting local events impacting atmospheric conditions such as a forest fire within a defined proximity to the user that is emitting harmful gases, waterline contamination or high level of toxins in drinking water, etc.). In step 614, the user closes the mobile platform. With reference to step 616, the platform can passively operate by recording predetermined elements even after the user closes the platform. For example, based on the five datasets, the mobile application repeats data cycling from a number of sources automatically, but may only elect to store specific data once the mobile platform recognizes the onset of a user's symptoms. In step 618, based on the analysis of the data subsets, the mobile platform may provide recommendations to the user to modify the user's actions such as by making a suggestion to reduce the sun exposure by limiting the time in the sun at the particular moment by exiting the area and returning at a time of day when the sun is not as strong and direct. It is further contemplated in one embodiment that the recommendation supplied by the mobile platform may be a sourced advertisement such as skincare manufacturer that offers a skin lotion of a strong SPF, further including a mobile link to allow the user to purchase the lotion. Advice specific to a user is generated based on the genetic attributes of an individual and the susceptibility to a specified concern associated with the user profile. By individually assessing the genetic risks of the user, specific risk factors can be identified and health advice, such as dietary recommendations, can be tailored to the user's needs.

FIG. 4 depicts a graph 700 of exemplary information stored in a user profile of mobile platform 100. User profile 702 is for the user named “Jane Doe” located at the address: 1 Main St., City, State, Postal Code, Country. User profile 702 includes at least one user data element 704. User data elements inputted by the user are represented in solid lines. Here, the user has inputted their age. User data elements with dashed lines represent elements that predictor processor 208 of mobile platform 100 has predicted based on the available information. Each predicted user data comprises predictor index 706. In this embodiment, predictor index 706 represents the likelihood that a predicted user data element is present. Further, when the predictor index is equal to or exceeds a threshold value, mobile platform 100 can provide notification of a predicted user data that is a risk factor. While the value of predictor index 706 can vary between 0.01 to 0.99, it would be readily apparent to one of ordinary skill in the art to use various scales for the predictor index to represent the likelihood of the presence of a user data element.

As shown in FIG. 4, the predictor processor has predicted that there is a pollen risk for the user, although the user has not indicated the presence of pollen at their location. Mobile platform 100 can access content providers 108 and application sourced data (i.e., the user's location information) for information on the pollen levels at the user's current location. Further, as described below, mobile platform 100 can utilize the information of other user profiles to determine a correlation between allergic reactions to pollen within the user's current location.

Another object of the preferred embodiment is to utilize user-generated data. The user-generated data is preferably user-inputted data. Exemplary user-inputted data includes, but is not limited to, food/beverage consumption, duration and type of exercise, time spent at work and type of work, presence of insomnia, nightmares, other sleep disorders, current or chronic mood, stress level, tension, and associated autonomic nervous system activity, sexual activity and sexual dysfunctions, time spent in sedentary activities such as watching television and movies, chronic conditions such as cardiovascular disorders, diabetes, and respiratory disorders. The user can also specify at least one health condition of interest. This information can be inputted manually, or the user can select a health condition from a predetermined list of conditions. In one embodiment, the user can enter positive experiences and events. Thereafter, mobile platform 100 can notify the user of similar activities related to the positive experience and/or event.

While the mobile platform can identify risk factors and predict health-related conditions with minimal input from the user, in some embodiments, the speed of the analysis can be improved by the user providing user-generated data. Some user data can also be generated by continuous, continual, or intermittent assessment of physiological responses, as measured by wearable devices such as a smart watches.

Yet another object of the preferred embodiment is the use of application-sourced data to track the user's location by means such as GPS and triangulation or trilateration of the user's cellphone. Other application-sourced activity involves the accessing and tracking of local environmental data, including but not limited to elevation, weather (barometric pressure, temperature, cloud cover, humidity, wind, etc.), moon phases, pollen count, air-borne particulates, activity (time spent in motion or inactive), and phone usage. The mobile platform periodically updates the stored data. In one embodiment, the application-stored data can includes physiological responses such as heart rate collected from wearables.

The mobile platform preferably allows the user to submit and update symptoms and health conditions. The mobile platform processes any updates or additional submissions according to the information stored on the user profile. For example, a user can submit a migraine six months after loading the application. Thereafter, the mobile platform correlates all the stored application-sourced and user-generated data with the new submission. As a result, a relevant diagnosis concerning the user's new submission can be quickly generated.

In some embodiments, data may be stored on the mobile platform or remotely. In an embodiment where the data is stored remotely, the mobile platform preferably continually communicates with the remote database. Therefore, the amount of data stored for a user profile can be dynamically increased or decreased by adding or removing additional remote databases. In this embodiment, the user can seamlessly retrieve or access information stored on the mobile platform and/or the remote database.

The application can also automatically access the user's heart rate, blood pressure, galvanic skin response (GSR), and other physiological data by linking to a wearable computer, such as “chipped” clothing, a smart watch, or an optical head-mounted display (OHMD) having means for receiving, processing, storing, and transmitting data. These exemplary wearable devices and others can contact the user's skin to facilitate the measurement of physiological data.

The mobile platform analyzes the symptoms, user-generated data, and application-sourced data to identify risk factors and predict health-related conditions.

Another object of the preferred embodiment is to determine the cause or causes of, or risk factors involved in, various health conditions. The health conditions include, but are not limited to, migraines, headaches, allergic reactions, psoriasis outbreaks, depression and other mental health conditions, asthma and other respiratory disorders, seizures and other neurological disorders, sexual dysfunctions, and cardiovascular symptoms and disorders.

The application can also include a diary function in which the user indicates a point in time when an incident occurs. Thereafter, the mobile platform can mark the time and correlate the diary entry with all data related to the point in time (application-sourced data).

Further, the mobile platform can cross-reference and analyze all of the information related to the user profile (i.e. user-generated data and application-sourced data) to determine recurring instances of combinations of factors that may be influencing and/or causing a health condition.

In some embodiments, the mobile platform can alert the user to the possibility of various health conditions unknown to, or unsuspected by, the user. The mobile platform can do so by correlating user inputted symptoms with ongoing collection of physiological data and accessing of information about environmental conditions.

Based on the information the application has collected, the application may need some additional information to “fill in the blanks” for diagnosis of a certain health condition. When the application recognizes the need for additional information from the user, the application can request additional information from the user as to whether he or she is experiencing a given symptom or group of symptoms.

In one embodiment, the data collected by the mobile platform is shared anonymously with an online community, thereby allowing each user's data to be compared with other users' data to help predict future outbreaks within a given area or venue, and also to aid medical research, such as epidemiological research.

For example, the mobile platform can permit users to observe that certain venues, neighborhoods, cities, or selected geographic ranges are dense with certain health conditions, whereas others are not. Users can be alerted when others in their area have indicated that they are experiencing an outbreak of a shared health condition. In one embodiment, a user can access a map showing locations/instances of other users with similar health conditions. Each data point can be marked as a dot on the user's display. Further, the dots can be differentiated using color coding to identify multiple heath conditions that the user is tracking.

In an exemplary display method, the user can decrease or enlarge the size of the geographic area covered by the display by compressing or spreading the display with the user's fingers. When the user makes the items on the screen smaller, thereby focusing on a larger area, the dots may merge. When the user enlarges a specific area, for example by spreading fingers on the screen, the dots can be displayed discrete. A user can interpret a concentration or density of health conditions as a risk factor for the health problem that has been listed and whose instances are being displayed. While the concentration of various users with similar health conditions in an area may not in itself reveal cause and effect, the mobile platform allows users to decide whether or not to frequent locations with a high density of their health problem.

Users can communicate with other users within the online community who may be experiencing similar symptoms or health conditions. The user can contact another user of the application by, for example, touching a dot on his or her display. Various possibilities for communication are possible. In one embodiment, a window can open for texting. In some embodiments, a user may send an email or transmit messages simultaneously to all users within the geographic location or venue shown on the display. When a message is sent, the user's profile or a part thereof, such as a photo, may accompany it. The user receiving the profile can in turn send their profile. The possible color-coding for a plurality of health conditions can also convert to meta-data that indicates which health condition the user is addressing. Other users—in this case, recipients of messages—can set their devices to accept or not accept messages from other users. Openness to reception of messages can be set as permanent unless changed, or to apply within a certain time frame or at a given location. Further, users can also block messages from a particular user, but the recipient would remain as a dot on the user's display. In this embodiment, the anonymity of a user is preserved unless the user decides to reveal their identity, photo, or certain kinds of demographic and profile information. Although one user may, for example, text another when a window for texting opens, the texting user would not know the phone number of the recipient. However, nothing prevents the users from exchanging contact and other information when windows of communication have been opened.

It may also be the case that those whose dots are displayed can be considered “followers” of the user, enabling the user to “tweet” or otherwise transmit messages to the grouping. The radius of the pool of recipients could be determined by the mobile platform or by the user.

Users can observe that persons with certain health problems are or are not likely to frequent certain restaurants. In one embodiment, the mobile platform can generate a screen that displays a summary of health issues afflicting users that frequent a restaurant. Based on the health profiles of users that utilize the mobile platform, users may be able to observe restaurant scores for allergies and migraines.

When users enter a condition, such as a current migraine, they can observe a recent map of their movements, barometric pressure, humidity, and so on. It may also be possible to enter dietary information, and the application can arrive at a solution in which it informs the user that he or she is most likely to experience a migraine when he or she has been in a certain neighborhood, in a certain type of weather, has eaten Chinese food or drunk red wine, and so forth.

The system and method disclosed herein are not limited to use by individual users. For example, the system can also be used for epidemiological research by health professionals. Health professional can use the information collected by the mobile platform to track neighborhoods or other venues that are associated with various health problems, the time of day that these problems are most pervasive, the barometric pressure, and pollen counts.

Further, some information collected by the mobile platform can be sold to various venues, such as stadiums, means of mass transportation, institutions, buildings, and restaurants. Restaurant owners, for example, may wish to know how their restaurant scores according to certain health factors in order to determine steps to improve their scores—for example, modifying the menus or physical environment—and become more attractive to their patrons.

In one embodiment, the mobile platform can be used to alert users when user-generated data and application-sourced data, including environmental data, predict the occurrence of a health condition such as an allergic response or a migraine headache. For example, the application may predict the occurrence of a migraine based on the user's location (GPS), recent alcohol intake, barometric pressure, ambient temperature, and the prevalence of the presence of other users with migraines. The user may also enter prodromal symptoms and rate their severity. In another embodiment involving wearable assessment devices, such as a smart watch, input variables such as heart rate, blood pressure, and galvanic skin response can also be used in the predictive matrix.

An embodiment of the mobile platform can be used to enable users to observe the prevalence of given symptoms and health conditions within a chosen venue, geographic range, or location (GPS). For example, users can observe whether other users are experiencing those symptoms and health conditions within the venue, range, or location. As a result, users may thus plan walking or other modes of travel routes that evade or circumscribe areas that are densely populated with individuals with the given symptoms and health conditions.

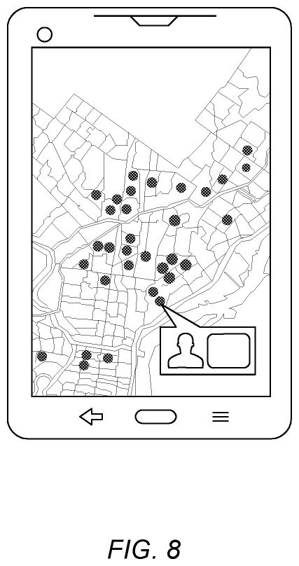

In another embodiment, the present invention can be used to enable users to select and communicate with other users in an area with similar symptoms or health conditions. In one embodiment shown in FIG. 8, other users are displayed as “dots” or similar entities on the user's display. By touching a given dot, a window for sending a message may be opened. In this embodiment, users can communicate only with other users with similar symptoms or health conditions. In initiating communication, the user may transmit some profile information such that the recipient can determine whether or not to respond to the message. Such profile information may be limited to a photo or may be as extensive as some social networking profiles. If the recipient responds, the recipient can transmit profile information.

An embodiment of the mobile platform can be used to enable users to transmit information concerning symptoms and health conditions, or venues in which these symptoms and health conditions are aggravated, to other users in the area. For example, users with food allergies could enable an alert that a given restaurant uses monosodium glutamate (“MSG”).

In one embodiment, the mobile platform can be used to compile for users a list of venues that are likely or unlikely to aggravate certain symptoms and health conditions based on the history of users' identification of such venues as locations in which their symptoms or health conditions appeared. These venues could appear on the user's display along with the display of other users who report given symptoms or health conditions. This embodiment can be used to compile scores for businesses or venues in an area based on user reports of, for example, aggravation of food allergies, other allergies, or migraines connected with proximity to or time spent within those businesses or venues. Businesses or venues can purchase information compiled by users as to the experiences they broadcast to other users. As described above, restaurant owners, for example, may wish to know how their restaurant scores according to certain health factors in order to determine steps to improve their scores—for example, by modifying the menus or physical environment—and become more attractive to their patrons. Exemplary user reports can be presented as anecdotal, numerical, or both. In this embodiment, the communications between individual users are not available.

In another embodiment, the principles disclosed herein can be used in medical/epidemiological research. For example, researchers can analyze neighborhoods or other venues associated with targeted health problems to compare environmental factors (temperature, barometric pressure, pollen counts, and the like) and summaries of user-generated data (age, gender, reported symptoms, BMI, blood pressure, and the like).

While the preferred embodiment has been set forth in considerable detail for the purposes of making a complete disclosure of the invention, the preferred embodiment is merely exemplary and is not intended to be limiting or represent an exhaustive enumeration of all aspects of the invention. It will be apparent to those of skill in the art that numerous changes may be made in such details without departing from the spirit and the principles of the invention. It should be appreciated that the present invention is capable of being embodied in other forms without departing from its essential characteristics.

What is claimed is:

1. A computer-implemented method comprising the steps of:
storing in a database a user profile, the user profile including at least one user data element;
monitoring at least one content provider;
gathering information from the at least one content provider related to a plurality of health risks;
predicting at least one risk factor based on the at least one user data element and the information gathered from the content provider; and
sending a notification comprising the at least one risk factor.

2. The computer-implemented method of claim 1, wherein the step of predicting the at least one risk factor comprises:
accessing information from the at least one content provider;
accessing at least one user generated information; and
correlating the information from the at least one content provider and the at least one user generated information.

3. The computer-implemented method of claim 2, further comprising the step of assigning a predictor factor to the at least one risk factor.

4. The computer-implemented method of claim 3, further comprising the step of assigning a value to the predictor factor between 0.01 to 0.99.

5. The computer-implemented method of claim 4, further comprising the steps of:
comparing the predictor factor of the at least one risk factor to a threshold value; and
sending a notification comprising the at least one risk factor comprising a predictor factor greater than the threshold value.

6. The computer-implemented method of claim 1, further comprising the step of displaying at least one user profile with a substantially similar medical condition.

7. The computer-implemented method of claim 6, further comprising the steps of:
displaying a map comprising a plurality of dots wherein the plurality of dots represent a plurality of user profiles with a substantially similar medical condition;
selecting at least one of the plurality of dots; and
viewing a user profile with a substantially similar medical condition.

8. The computer-implemented method of claim 3, further comprising the step of:
allowing users with a substantially similar medical condition to communicate.

9. The computer-implemented method of claim 1, further comprising the steps of
calculating a user location; and
recommending a service provider based on the at least one risk factor, a location of the service provider, and the user location.

10. The computer-implemented method of claim 9, wherein the step of calculating a user location comprises utilizing a Global Positioning System.

11. The computer-implemented method of claim 10, further comprising the step of collecting physiological data utilizing a wearable device.

12. The system of claim 11, wherein the communication module is configured to communicate through the network a notification to the at least one mobile platform terminal comprising the at least one risk factor.

13. The system of claim 11, wherein the user profile comprises at least one user data element comprising a predictor index.

14. The system of claim 13, wherein the predictor index comprises a value between 0.01 to 0.99.

15. A system comprising:
a mobile platform;
a network;
at least one content provider;
at least one mobile platform terminal;
at least one service provider;
wherein the mobile platform communicates a notification to the at least one mobile platform terminal comprising a risk factor.; and
wherein the content provider comprises information related to a plurality of health risks.

16. The system of claim 15, wherein the mobile platform comprises:
a CPU;
at least one memory;
a predictor processor;
a communication module;
at least one user profile database comprising at least one user profile; and
at least one service provider database comprising at least one service profile;
wherein the predictor processor is configured to predict at least one risk factor for the at least one user profile.

17. A computer-implemented method, in a computer having a processor and a memory coupled to the processor, comprising:
monitoring a plurality of user profiles comprising at least one user data element;
monitoring at least one content provider;
gathering information from the at least one content provider related to a plurality of health risks;
predicting at least one risk factor by correlating information received form the at least one content provider and information received from the plurality of user profiles;
generating a notification to the plurality of user profiles comprising the at least one risk factor.

18. The computer-implemented method of claim 17, wherein the at least one risk factor is a substantially similar medical condition of the plurality of user profiles.
